(19) United States
(12) Patent Application Publication
(10) Pub. No.: US 2005/0203553 A1
(43) Pub. Date: Sep. 15, 2005

(54) Device for the performance and monitoring of rotablation

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/055,868

(22) Filed: Feb. 11, 2005

(30) Foreign Application Priority Data
Feb. 20, 2004 (DE) 10 2004 008 370.3

(51) Int. Cl. A61B 17/22

(52) U.S. Cl. 606/159

(57) ABSTRACT

Device for the performance and monitoring of rotablation, wherein a rotating bore disposed at the tip of a catheter removes plaque deposited on the vascular wall while deflecting normal vascular tissue, a rotablation catheter and an OCT catheter being integrated to form a constructional unit.

The invention relates to a device for performing and monitoring rotablation, wherein a rotating burr disposed at the tip of a catheter removes plaques deposited on the vascular wall while deflecting normal vascular tissue.

One of the world's most common fatal diseases are vascular diseases, in particular myocardial infarction. This is caused by arteriosclerosis whereby deposits (arteriosclerotic plaque) result in a blockage of coronary vessels. When coronary angiography indicates severe narrowings (stenoses) in the coronary vessels, causing angina pectoris and limiting functional capacity and/or threatening the patient, in the majority of cases PTCA (percutaneous transluminal coronary angioplasty) is nowadays performed. For this purpose the narrowed parts of the coronary vessels are dilated using the so-called “balloon catheter”.

Clinical studies have shown that with this method restenosis occurs in many patients, in some cases up to 50% of patients exhibit restenoses. For some years an alternative method for removing the plaque has therefore been increasingly used, so-called high frequency rotablation angioplasty, which offers advantages particularly in the case of severely fibrotic or calcified and/or long-segment stenoses.

Coronary rotablation angioplasty is a so-called “debulking” system (recanalization of stenosed coronary arteries).

The rotablation angioplasty system consists of a diamond-coated burr which rotates at very high speed and selectively removes calcified and fibrotic plaques, the normal elastic vascular wall being deflected away by the burr and remaining undamaged (“differential cutting”). The resulting microparticles are flushed out to the periphery. The method has established itself as a valuable instrument for severely calcified lesions which cannot be removed by simple balloon angioplasty. In contrast to balloon angioplasty, the stenosis is not dilated. At a typical rotation speed of 150,000 rpm the trimmed microparticles are so small that they are filtered by the liver, lung and spleen without causing damage in the body.

A device for rotablation angioplasty is described, for example, in U.S. Pat. No. 5,356,418, in EP 0 794 734 B1 and in EP 0 267 539 B1. The “device for transluminal microdissection” described in EP 0 267 539 B1 is essentially the Boston Scientific product known under the name Rotablator®.

The rotablator consists of a burr with a diameter of approximately 1-3 mm which is connected via a highly flexible shaft to a pneumatically driven turbine (typical speed 20,000-155,000 rpm). The turbine is driven by compressed air and controlled via a console which is activated using a foot pedal.

The flexible shaft comprises the drive cable and is surrounded by a Teflon sheath through which a rinsing fluid is forced. The rinsing fluid prevents heating of the drive cable as well as ensuring that the microparticles are washed away to the distal end. The shaft with the burr can be replaced without having to replace the turbine. The approximately 3 m long and thin (approx. 0.2-0.3 mm) guide wire (“RotaWire™”) over which the drilling probe is pushed is automatically locked in the turbine during rotablation. However, this locking can be released so that the burr and the wire can be moved independently of one another. This is frequently used in order to withdraw the burr from the coronary artery.

The therapy described above is performed using angiography equipment under x-ray control by means of a contrast medium. The disadvantage of this method is that the coronary vessels are only visualized two-dimensionally and only the actual narrowing appears in the x-ray image. During the operation, medical personnel have difficulty differentiating between plaque and vessel wall. The purely angiographic assessment of the severity of calcification and in particular of the position of the calcium in the plaque (superficial versus deep) is difficult. This means a considerable risk for the patient, as either too little plaque is removed and the desired blood flow is not restored or the risk of restenosis remains, or too much tissue is removed, possibly resulting in perforation of the vessel.

In order to make the plaque more clearly visible, a separate intravascular ultrasound (IVUS) catheter could be introduced into the vessel. An IVUS system is described, for example, in DE 198 27 460 A1 and in U.S. Pat. No. 5,193,546. Or a separate OCT catheter could be introduced into the vessel. The OCT method is described, for example, in WO 01/11409 A2 (LightLab), in U.S. Pat. No. 5,921,926 and in EP 0 815 801 B1. OCT operates in a similar manner to imaging ultrasound (B-mode). The underlying physical principle is based on the Michelson interferometer.

The disadvantage of this approach is that the entire rotablation device must be withdrawn from the vessel each time.

U.S. Pat. No. 5,312,427 describes a device having a double-lumen catheter whereby one lumen can be used for introducing an IVUS probe. The disadvantage of this solution lies in the double-lumen catheter which must have a much larger diameter than normally used catheters and is therefore poorly suited for introducing into coronary arteries. The further disadvantage of this solution lies in the increased rigidity of the catheter due to the double lumen. Another disadvantage of this solution is the decentralized position of the introduced IVUS probe relative to the rotablator burr.

The object of the invention is therefore to create a device for simplified performance and monitoring of rotablation wherein precise observation of the target area and intervention to remove the plaque are simultaneously possible without changing catheters.

This object is achieved according to the invention by integrating a rotablation catheter with an OCT catheter (optical coherence tomography catheter) to form a constructional unit, preferably implemented in such a way that the OCT line, preferably implemented as a glass fiber line, runs to the OCT sensor (which is connected to the burr and is in turn preferably implemented as a rotating mirror) in a highly flexible drive shaft which drives the burr and the OCT sensor in a rotating manner.

The inventive combination of an OCT catheter with a rotablation angioplasty catheter to form an integrated unit results in an optimum system for “debulking” coronary vessels. The great advantage of this solution is that it reduces both process steps and catheters used, as well as reducing the x-radiation applied. The images of the OCT system provide important additional medical information about the plaque and the vascular wall, e.g. inflammatory processes. The OCT method (optical coherence tomography) is described e.g. in WO 01/11409 A2, U.S. Pat. No. 5,921,926 and EP 0 815 801 B1. OCT operates in a similar manner to imaging ultrasound (B-mode). The underlying physical principle is based on the Michelson interferometer. It enables the blocked vessel section to be better detected in each case and the removal of the plaque to be monitored during and after the procedure. The particular advantage of the OCT method is the very high detail resolution of structures near the vessel surface, which in some cases makes microscopic tissue visualizations possible.

According to another feature of the invention it can be provided that a micro gear is interposed between the burr and the OCT sensor so that the burr can rotate at a speed independent of the rotation of the OCT sensor. The catheter sheath can advantageously be provided with end inlet or outlet openings for contrast medium or rinsing fluid, as the use of an OCT catheter makes it necessary to inject a flush solution (e.g. physiological saline) in the region of the site under examination.

In addition to disposing magnets at the catheter tip for magnetic navigation, the device can also be implemented with a guide wire passing through it.

Finally it is also within the scope of the invention that an inflatable, preferably multi-chamber balloon for fixing the catheter in position and/or used for vessel dilation is disposed at the catheter tip.

Further advantages, features and details of the invention will emerge from the following description of an exemplary embodiment and with reference to the accompanying drawings in which:

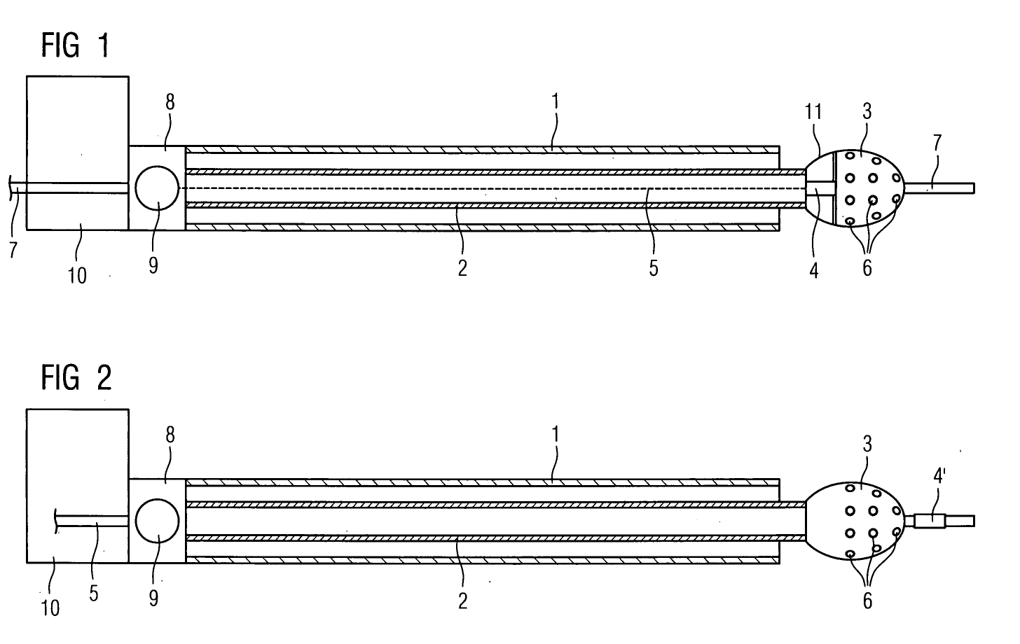

FIG. 1 shows a schematic cross-section through a combined OCT-rotablation catheter according to the invention, wherein the OCT sensor is disposed behind the actual cutting section of the burr, and

FIG. 2 shows a modified embodiment of such a combined OCT-rotablation catheter with OCT sensor disposed ahead of the burr.

The combined OCT-rotablation catheter shown in FIG. 1 comprises a catheter sheath 1 in which there is disposed a hollow flexible drive shaft 2 which is used for driving both the burr 3 and the OCT sensor 4 (rotating mirror) disposed in its rear section within a preferably revolving window 11. Reference character 5 designates a glass fiber line forming the signal line to the OCT sensor 4. The front section of the burr 3 is coated with abrading/cutting particles 6 which are implemented in such a way that they deflect normal vessel tissue away during rotation and only remove plaque deposited on the intravascular wall. Reference character 7 designates a guide wire running through the catheter, but not shown in the middle for the sake of clarity, which is first inserted into the vessel being treated as far as the target area prior to introduction of the combination catheter, the combination OCT-rotablation catheter according to the invention then being pushed onto the guide wire and advanced to the target region. Both the introduction of the guide wire 7 and the introduction of the burr with the integrated OCT-rotablation catheter take place under x-ray control, possibly using a contrast medium. Using the OCT probe, the location at which the plaque is to be removed is examined more precisely (during this examination the combination probe rotates at relatively low speed, e.g. approximately 100 to 1,500 rpm), a rinsing fluid being simultaneously injected for the OCT process. The burr is then slowly moved into the stenosis at high rotation speed and is gently withdrawn after a few seconds. When a certain amount of plaque has been removed, the location on the vessel wall is inspected with the OCT sensor. The process is repeated until the plaque has been removed at all the locations.

In addition to the mechanical linkage system 8 and the rotary coupling 9 for the connections, there is also provided a signal interface and drive unit 10 for operating the combination sensor. There are additionally provided the abovementioned feed and drain lines for the rinsing fluid which, however, are not included in the drawing for the sake of clarity.

The modified version of a combined OCT-rotablation catheter according to FIG. 2 essentially differs from that shown in FIG. 1 only in that the OCT sensor is not provided in the burr behind its cutting particles, but preceding it at 4′ and that the hollow flexible drive shaft 2 is provided with an integrated lumen for the passage of the OCT sensor.

In both embodiments, in particular a micro gear can be interposed between the burr and the OCT sensor in addition to a magnet in the catheter tip for magnetic navigation in order to be able to operate both at different speeds.

A medical system comprising combined OCT-rotablation angioplasty catheter and subsystem for connecting the OCT-rotablation angioplasty catheter consists of a signal interface unit, preprocessing for OCT image data, and an image processing and visualization unit. It also includes a user interface for controlling the system and for operating the visualization for OCT including image memory, voltage supply unit and network interface (e.g. DICOM), as well as a drive unit for the hollow flexible drive shaft. The drive unit is capable of providing the high speed (e.g. 150,000 rpm) for the burr and also the low speed (approximately 1,000 rpm) for the OCT probe. At the low speed for the OCT probe, a relatively constant speed is necessary, so that it is advisable for the high-speed to be produced, as noted, with a compressed air driven turbine, while the low speed can be produced with a regulated electric drive.

The OCT imaging system can be upgraded to include menus in order to allow quantification (e.g. measurement of angles, lengths, surfaces, stenosis rate before and after the procedure) of the stenosis and of the removed plaque.

Finally, it would also be possible—in addition to using conventional x-ray markers on the catheter shaft—to mount a temperature sensor at the tip of the catheter (not shown in the embodiment illustrated) in order to check the heat due to friction at high speeds. Clinical studies have shown that heat damage in the vessels increases the rate of restenosis.

1-8. (canceled)

9. A device for applying and monitoring medical rotablation, comprising:
a first catheter sized and configured for medical rotablation;
a second catheter sized and configured for OCT-monitoring, the first and second catheters integrated into one catheter unit having a catheter tip; and
a drill head arranged adjacent to the catheter tip for removing plaque from a vascular wall, the drill head adapted to deflect normal vascular tissue while removing the plaque.

10. The device according to claim 9, further comprising:
an OCT sensor operatively connected to the drill head;
an OCT signaling line connected to the OCT sensor; and
a hollow, flexible drive shaft for rotating the drill head and the OCT sensor, wherein the OCT signaling line is arranged within the drive shaft.

11. The device according to claim 10, wherein the OCT signaling line includes an optical fiber.

12. The device according to claim 10, wherein the OCT sensor includes a rotating mirror.

13. The device according to claim 10, further comprising a micro gear unit operatively connected to the drill head and the OCT sensor, the micro gear unit arranged downstream of the drill head and upstream of the OCT sensor.

14. The device according to claim 9, further comprising a catheter jacket having provided inlet or outlet openings for feeding a contrast medium or rinsing fluid to respectively discharging the contrast medium or rinsing fluid from the device.

15. The device according to claim 9, further comprising a plurality of magnets arranged at the catheter tip for enabling magnetic navigation of the device.

16. The device according to claim 9, further comprising a continuous guide wire.

17. The device according to claim 9, further comprising an inflatable balloon arranged at the catheter tip for locating the catheter at a desired position and/or for dilating a vessel.

18. The device according to claim 17, wherein the balloon comprises a plurality of inflatable chambers.

19. The device according to claim 9, further comprising a temperature sensor arranged at the catheter tip.
